(19) United States
(12) Patent Application Publication
(10) Pub. No.: US 2016/0278615 A1
(43) Pub. Date: Sep. 29, 2016

(54) WIRELESS ENDOSCOPIC SURGICAL DEVICE

(71) Applicant: Scopernicus, LLC, Cranford, NJ (US)

(72) Inventors: Paul John Kawula, Sunnyvale, CA (US); James McAvinn, Stanhope, NJ (US); Patrick T. Walsh, Cranford, NJ (US)

(73) Assignee: Scopernicus, LLC, Cranford, NJ (US)

(21) Appl. No.: 15/094,992

(22) Filed: Apr. 8, 2016

Related U.S. Application Data
(63) Continuation-in-part of application No. 13/759,920, filed on Feb. 5, 2013, now abandoned.

(51) Int. Cl. A61B 1/00; A61B 1/018; A61B 1/07; A61B 1/06; A61B 1/04

(52) U.S. Cl. CPC A61B 1/00016; A61B 1/00066; A61B 1/00032; A61B 1/0669; A61B 1/042; A61B 1/07; A61B 1/0684; A61B 1/00009; A61B 1/018; A61B 1/00045

(57) ABSTRACT

A wireless endoscopic surgical device used for minimally invasive procedures comprises a handheld component and a separate power module. The handheld component consisting of a handle and a conduit houses a wireless imaging system and a single LED light source. The imaging system comprises a wireless camera coupled to an optical assembly. Both the intensity of the LED and the camera action can be controlled by a battery-operated power module. The handle and conduit are designed to accommodate surgical tools. In alternative embodiments, the handheld component is self-contained.

### REFERENCE TO RELATED REFERENCES

This application is a Continuation-in-Part of U.S. patent application Ser. No. 13/759,920, filed on Feb. 5, 2013, the contents of which are hereby incorporated by reference in their entirety.

### BACKGROUND

Expensive medical devices are typically reused. The portions of those devices that contact tissue are sometime shielded with a disposable cover or sterilized after use. This concept is well known. The medical device in question would be called reusable.

But as far as patient safety is concerned, reusable devices frequently pose a greater infection risk than disposable medical devices. Using a shield, as described above, transfers some of the advantages of disposables to the reusable device, but also adds complications to its operation.

In many cases, a disposable device with similar performance to the reusable device would have a competitive advantage. For purposes of this disclosure, a “disposable” device or a device said to be “disposable” is defined as a device that is used once for a procedure and then discarded such that those of ordinary skill in the art would view discarding the device as reasonable in view of the overall benefits from avoiding reuse of the device.

Thus, while any medical device could be discarded after a single use, in some cases doing so would be unreasonable to those of ordinary skill in the art.

Historically, endoscopes are reusable devices. Endoscopes are used to view the inside of the body through a small incision during minimally invasive surgery.

A rigid endoscope system comprises the following: the endoscope itself, that is, a long tubular metallic conduit that contains optics that extend from the proximal end in a handle to the distal viewing tip. A light source cable connects to the proximal end to provide light for viewing, and the resultant image is carried through a separate optical system (lenses), back to an external camera at the proximal end. Images may be processed and stored in the camera or sent to a monitor for viewing, after being processed in an external video processing box.

Endoscopes can have issues: first is failure of a component of a system, especially if it is a re-processable item; and second is the bulk or unwieldy nature of a system.

Endoscopes are delicate instruments, and can become damaged with repeated use, cleaning, or resterilization. Owing to the cost, most cardiac operating rooms (ORs) do not have many back-up scopes.

Optics are important parts of endoscopes. But aside from improving optical image quality, the essential elements of what is used for transferring light from the source to the target and the resultant image back to the camera have not changed much over time. Light and images are transferred by combinations of fiber optic bundles, lenses and mirrors.

Fiber optic bundles can be cost effective. But they can display optical artifacts from packing density that can worsen with length. For this reason, many rigid endoscopes, gradient-index (GRIN) lenses have been used. But GRIN lenses are long, rigid lenses, limited in the length they can be made, and are historically costly.

Ergonomic or logistic problems frequently seen in the OR suite stem from having many wires. As the wired devices are used during the procedures, the wires inevitably entangle with each other. Frequently, such tangling causes surgical components to break during the procedure, causing an FDA reportable incident. In some surgery cases, the fiber optic light cable and camera power cord stretching from the equipment-laden tower to the patient table causes clutter and becomes a potential tripping more other safety hazard especially with many operators and technicians working in a small OR. draping cables and cords within the OR is an important reason that wireless connectivity within the OR is promoted.

Additionally, damage or failure in a scope discovered during system set up could trigger not only repair work, but if no back-up scopes were immediately available, could also force conversion to an open procedure. In Endoscopic Vein Harvesting (EVH), this also becomes an FDA-reportable incident requiring reporting and follow-up. An open procedure becomes a regular surgical procedure with associated cost and patient discomfort save it.

Therefore, endoscopes are cleaned, re-sterilized, and stored with great care. Scope use is tracked, and scopes are maintained and upgraded as necessary. Education and training in scope care as well as the actual cleaning expend staff time. Light source boxes for the scopes, although not as delicate, also need to be maintained as capital equipment. And this adds time and resource costs to hospital operation.

Also, in some cases tool lumens within the endoscope enter the endoscope body offset from the center of the endoscope. Offset entry can allow increased torque to inadvertently be applied to a tool, once again contributing to breakage. Moreover, offset entry requires redirection of the tip of the tool, which requires a redirection force usually provided by a plate inside of the device. The top of the surgical device can hang or catch on the plate. This interaction interferes with smooth surgical device operation. Sometimes the problem is related to a feeling of stiction within the device. In any case, offset entry interferes with the operator's use of the device. Also, the interaction of the tip of the surgical device and the redirection plate can grind material off of the tip or the plate. This material is frequently deposited in the patient.

Endoscope set up carries with it inherent safety issues. The external light source box can get hot and cause burns if mis-handled.

Even with functioning components, device assembly still takes time.

If some or all an endoscope systems were integrated and available to the operator as one device, some of these issues could be alleviated.

### BRIEF SUMMARY

Various invention embodiments supply an endoscope system with a self-contained endoscope. The endoscope can have a handle, a conduit having a proximal end connected to the distal end of the handle, a power and control module disposed within the handle, a light system disposed within the conduit and within the handle and electrically connected to the power and control module, an imaging system disposed with in the handle and electrically connected to the power and control module, and a video camera disposed within the handle optically connected to the proximal end of the imaging system and electrically connected to the power and control module.

In some of these embodiments, the light system employs coherent fiber bundles in one way or another. In these or other embodiments the light system employs an LED or a high intensity LED.

In some embodiments, the self-contained endoscope is battery powered and the power and control module comprises a battery. In some embodiments, a super capacitor supplies electrical power.

In some embodiments, the imaging system of the self-contained endoscope uses an RF receiver or transceiver. In these or other embodiments, the imaging system uses an optical data processing unit for compression, image enhancement or other processing as is known to those of ordinary skill in the art.

In some embodiments, the self-contained endoscope has a tool bore disposed at or along the central endoscope axis. In some embodiments, the light system is offset to allow space for the tool bore to pass through the endoscope. In these or other embodiments, the light system or the CF bundles of the light system coaxially lie around the imaging system. In these or other embodiments, folding the imaging system path or the light system path within the handle move these components to the outer portion of the endoscope likewise providing space for a central tool bore.

In some embodiments employ a discrete base having a receiver or transceiver and a display.

### BRIEF DESCRIPTION OF THE DRAWINGS

FIG. 1 is an overall system layout of an invention embodiment, showing the major components and their interconnections.

FIG. 2 depicts an embodiment of an endoscopic device (see FIG. 1).

FIG. 3 depicts an embodiment of the imaging assembly.

FIG. 4 provides two views of a distal lumen baffle 750 and an optically transparent shield 151.

FIG. 5 is a block diagram of the power and control module (PCM).

FIG. 6 depict an embodiment of a self-contained endoscope.

FIG. 7 depicts an embodiment of a self-contained endoscope having a bent or folded optical path in the imaging system.

FIGS. 8A-C show cross-sections of various embodiments of conduit 150.

### DETAILED DESCRIPTION

EN device 110
Housing 111
PCM 120
Cable 125
Receiver 130
Handle 140
Handle body 141
End cap 142
T-slot 143
Cutout 145
Tool port 146
Tool bore 147
Ventilation openings 148, 149
Conduit 150
Tip 151
Transmissive joint 155
Imaging system 160
Achromatic lens 161
Color camera 170
Camera sensor 171
Light system 180
Display 190
Distal CF bundle 201
Optic elements 203-208
Distal IA end 212
Distal Face 214
Proximal Face 215
Distal end 218
Dual-lens housings 209-211
Proximal CF bundle 221
Coupler 229
Focus 230
Antenna 235
LED 237
Finned heat sink 238
Light pipe 240
Wiring 243
LP tip 248
Imaging assembly 260
Distal IA end 261
Switch 302
Connector 307
Indicators 311, 312
Potentiometer 313
Shaft 314
Electrical system 315
Camera block 317
Battery holder 353
PCB 354
LS control 380
Mirrors 409, 410
Focal adjustment screw 572
Focal adjustment knob 573
Conduit seal 705
Lens washing system 710
Locking pins 715, 716
Locking slots 718, 719
Transparent shield 720
Cutouts 752, 740, 735, and 730
Distal lumen baffle 750
Optical data processing unit 774
Electrostatic shield 775
Power on/off switch 776

The following description of several embodiments describes non-limiting examples that further illustrate the invention. No titles of sections contained herein, including those appearing above, are limitations on the invention, but rather they are provided to structure the illustrative description of the invention that is provided by the specification.

Unless defined otherwise, all technical and scientific terms used in this document have the same meanings that one skilled in the art to which the disclosed invention pertains would ascribe to them. The singular forms “a”, “an”, and “the” include plural referents unless the context clearly indicates otherwise. Thus, for example, reference to “fluid” refers to one or more fluids, such as two or more fluids, three or more fluids, etc. Any mention of an element includes that element's equivalents as known to those skilled in the art.

Any methods and materials similar or equivalent to those described in this document can be used in the practice or testing of the present invention. This disclosure incorporates by reference all publications mentioned in this disclosure all of the information disclosed in the publications.

The features, aspects, and advantages of the invention will become more apparent from the following detailed description, appended claims, and accompanying drawings.

This disclosure discusses publications only to facilitate describing the current invention. Their inclusion in this document is not an admission that they are effective prior art to this invention, nor does it indicate that their dates of publication or effectiveness are as printed on the document.

For purposes of this disclosure, “discrete” means lacking a physical connection to another object. For example, an object resting on the desk would be considered to be discrete from the desk. But if a screw connected the object to the desk it would not be considered “discrete”. Likewise, if an object were resting on the battery it would be discrete from battery, but if it was connected to the battery with electrical wiring, it would not be discrete. For purposes of this disclosure, “self-contained” means having all of the components necessary for operation. For example, a self-contained medical device would contain all of the components necessary for operating the medical device within the device itself. For purposes of this disclosure, “isolated” means not physically connected to another component of the system.

For purposes of this disclosure, “reposable” devices are devices designed to have portions that are disposable and portions designed for reuse. In some versions of “reposable”, the device is designed such that components that are more readily cleaned or sterilized after use, while less readily sterilized or cleaned components are not necessarily designed for reuse. In some versions, the more expensive components are designed to minimize the difficulty of reusing or sterilizing the device. In some cases, reposable devices include devices having been designed to facilitate reconditioning. In some cases, reposable devices are designed for greater than 5 are 10 uses.

It is expected that the disclosed system will make procedures simpler for the operator and by extension make the patient more comfortable. The devices are also expected to provide large cost savings for the hospital as costly capital equipment (scope and light source) need not be maintained, and associated costs tied to reprocessing the scope (staff time, cleaning and sterilization costs) are eliminated.

The system largely dispenses with component assembly or attachment to outside equipment. ORs could keep an inventory of these systems for procedures. Should any damage be discovered, another package could be opened without delay in procedure or conversion to open surgery.

For those systems that are disposable, facilities (hospitals) would not need to educate staff members in special cleaning, sterilization, or maintenance procedures. This frees time and resources for the hospital. Single-use devices such as this also makes for simpler inventory control dispensing with coordinating capital equipment service agreements with vendors.

The internalized camera, wireless transmission of the image, and optics designed around a device configuration enabled the overall size of the device to be small. Compared with an assemblage of cannula, camera, and associated cables and cords of a conventional system, a conduit with a handle is much more compact and therefore expected to be easier for the operator to manipulate during the procedure.

### System Components

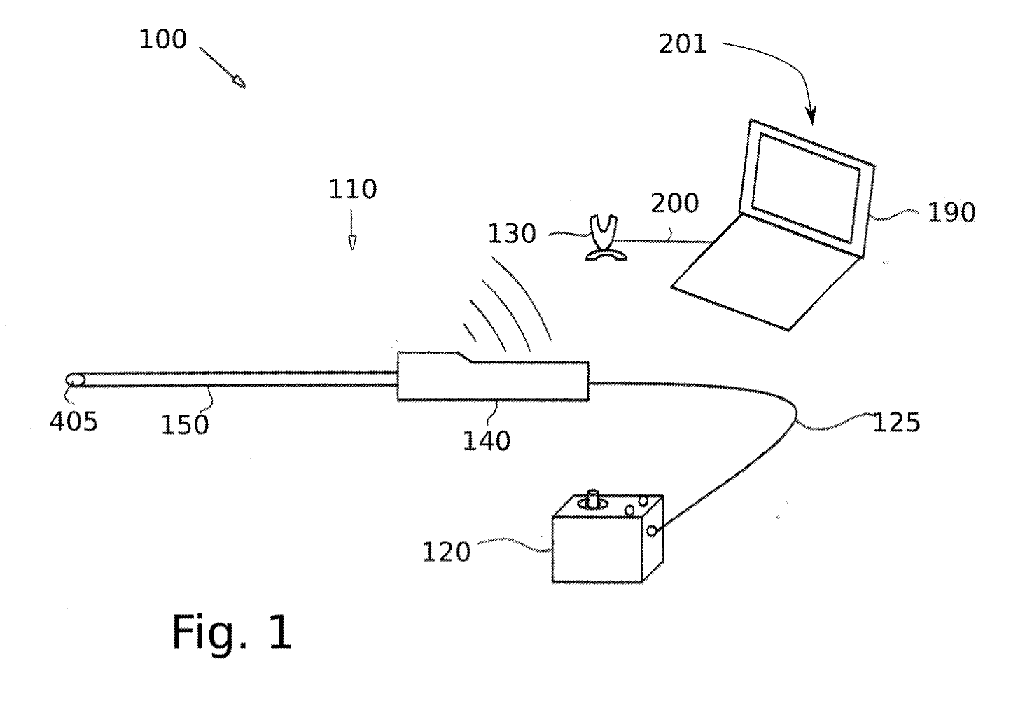

FIG. 1 shows an example of invention EN system 100. EN system 100 comprises EN device 110, cable 125, power and control module (PCM) 120, receiver 130, handle 140, conduit 150, monitor 190, and data cable 200. Imaging system 160, color camera 170, light system 180 are not shown in FIG. 1. In some embodiments, cable 125 is optional. In these or other embodiments, PCM 120 is part of handle 140 or is contained within housing 111.

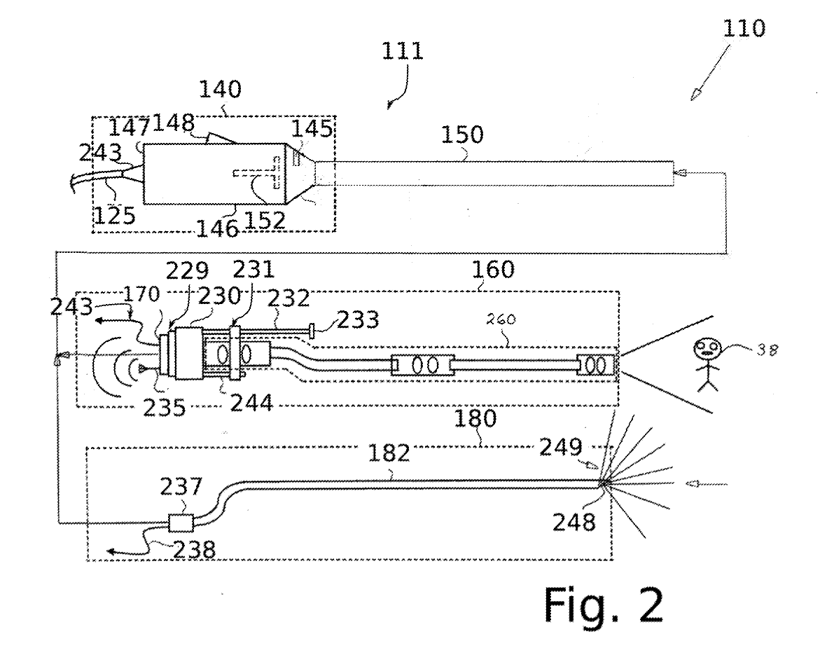

As shown in FIG. 2, handle 140 connects to conduit 150 to form housing 111. In some embodiments, the connection between handle 140 and conduit 150 allows for disconnection between these components, and in some embodiments the connection is permanent. Handle 140 also connects to PCM 120 through cable 125. This connection provides an electrical supply to handle 140.

In the current invention, a system is described that integrates light system 180 and imaging system 160 into a single conduit-150-handle-140 assembly. In some embodiments, the PCM 120 is also inside of handle 140. In other embodiments, cable 125 connects to PCM 120 to handle 140. In some embodiments, this integrated system is disposable.

Moving back to FIG. 1, one sees that base 201 comprises receiver 130 that, in some embodiments, has wireless connectivity with camera 170. Data line 200 connects receiver 130 to monitor 190 and transmits data from or to and from receiver 130 to monitor 190. In some embodiments, monitor 190 is a general or special purpose computer.

FIG. 2 shows an embodiment of the invention EN System 110. It depicts an embodiment of housing 111, and an embodiment of imaging system 160, and an embodiment of light system 180.

Housing 111 has handle 140, handle body 141, end cap 142, T-slot 143, cutout 145, and tool port 146. T-slot 143 is used in some embodiments to receive a manipulation tool (not shown). Cut-out 145 receives focus wheel 233.

In some embodiments imaging system 160 or light system 180 are disposed against the inside wall of conduit 150. Moving the imaging system 160 and light system 180 up against the outer wall of conduit 150 facilitates passing a surgical instrument down the center of EN device 110. In some embodiments, the surgical device is coaxial with the EN device 110, rotation of EN device 110 can occur while the surgical device remains stationary.

As depicted in FIG. 2, imaging system 160 comprises color camera 170, coupler 229, focus system 230, and image assembly 260. Imaging system 160 lies within housing 111. And coupler 229 connects focus system 230 to color camera 170.

Color camera 170 has wiring 243, antenna 235, and camera sensor 171 (not shown in FIG. 2). Color camera 170 converts light impinging on camera sensor 171 into electrical signals and transmits those signals through antenna 235. In some embodiments, color camera 170 receives electrical signals such as power or control signals through wiring 243. In some embodiments, camera sensor 171 is a high definition (HD) charge coupled device (CCD).

Suitable cameras are commercially available and well-known to those of ordinary skill in the art. Suitable cameras transmit image data using RF or free-space optical communication. In various embodiments suitable cameras transmit within the Industrial, Scientific, and Medical (ISM) frequency band. In various other embodiments, the cameras operate in the Wireless Body Area Network (WBAN) or 2.4 or 5.8 GHz band or the 900 MHz band. In some embodiments, the color camera 170 mounts in handle 140 and receives power from PCM 120. As those of ordinary skill in the art will recognize, other image detector technologies are useful in suitable color cameras

Printed circuit boards used in various invention embodiments are designed for specific refresh and scan rates, to match display 190. This delivers optimum performance, by preventing edge effects from mismatched formats within to the camera-monitor display. Additionally, sync signals from camera to PCM 120 can eliminate any power drop-out and/or disruptions that cause temporary signal loss or HD image loss at the monitor.

The embodiment in FIG. 2, has a wired color camera 170 with selectable resolution (1080P/30/720). This device also dramatically reduces the number of cords and external fiber optic illumination cables running to EN device 110. A single power line about 0.200″ in diameter leads to the back end of handle 140, for wired embodiments.

In some embodiments, the outer diameter of conduit 150 (a stainless steel tube) is about 0.5 to 5.2 mm. In other embodiments, such components are about 12.7 mm OD and comprise internal ports for assorted surgical tools. The outer diameter (OD) of conduit 150 is between 5.0 and 5.2 mm in diameter, in some embodiments. EVH-specific scopes sometimes use 12.7 mm OD and have internal ports for assorted surgical tools.

Focus system 230 comprises focus wheel 233, wheel shaft 232, plate 231, and alignment rod 244. Focus system 230 receives light representing an image at its distal end and focuses that image through coupler 229 onto an imaging plate or detector. Focus wheel 233 changes the length of the focal elements inside of focus system 230 to cause the image to come into focus. Those of ordinary skill in the art are experienced with the construction and selection of focusing systems for endoscopes.

As with imaging system 160, imaging assembly 260 lies within housing 111.

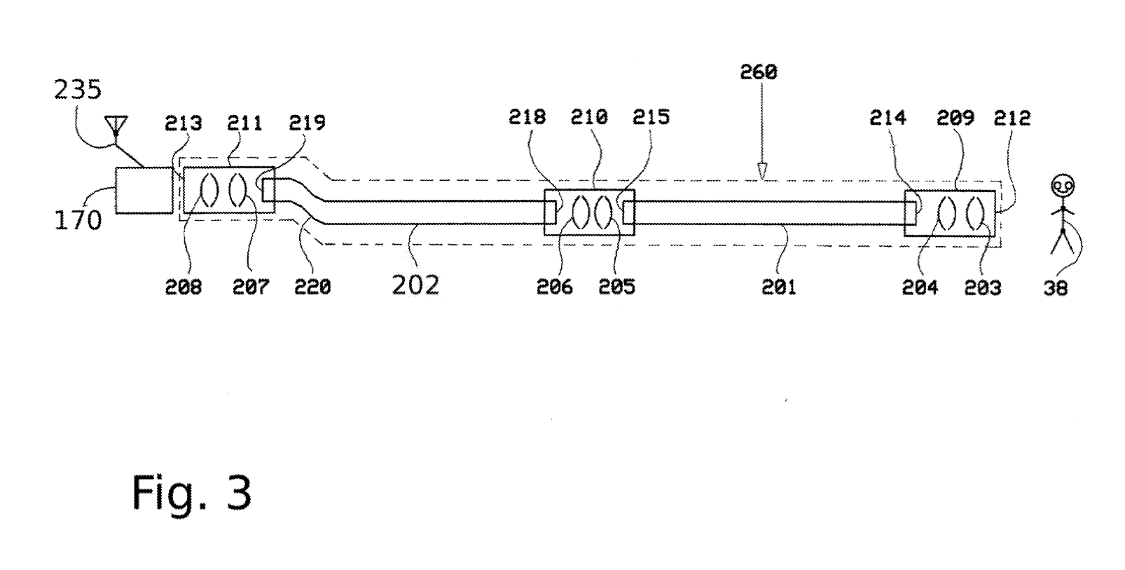

FIG. 3 depicts an embodiment of an imaging assembly 260 that is part of EN device 110. Imaging assembly 260 comprises two, segmented, coherent fiber (CF) bundles 201 and 221, six achromatic optic elements 203 through 208, and three dual-lens housings 209, 210, and 211. Segmented CF bundles (201 and 221) comprise fiber segments of a length and diameter appropriate to fit EN device 110 in FIG. 2. CF bundles (201 and 221) relay an image of the target 38 through close-packed fibers while maintaining image orientation. Each of the optic elements (203 through 208) comprise different classes and exhibit different grind radiuses to counter spherical and chromatic aberrations of the image. The image first impinges on distal IA end 212. Achromatic optic elements 203 and 204 lie within dual-lens housing 209 and transfer and focus the image at distal IA end 212 to distal CF bundle end 214. The number of optic elements, lens housings, etc. is exemplary only and will rise or fall as the optical design dictates.

Optic elements 203, 204 are contained at the distal end 212 of the imaging assembly 260. They transfer and collect an image of target 38 to distal face 214 of distal CF bundle 201. Distal CF bundle 201 extends from dual-lens housing 209 to dual lens housing 210. Distal CF bundle 201 transfers the image to proximal face 215 of distal CF bundle 201. Dual lens housing 210 has optic elements 205 and 206 The second two optic elements 205 and 206 are contained in the second dual-lens housing 210. These two optic elements (205 and 206) have focal lengths that project the image at proximal end 215 to distal end 218 of proximal CF bundle 221 without substantial distortion. This coupling technique is known as Free Space Optical Coupling.

Optic elements 207 and 208 are inside of dual-lens housing 211 and similar to the optic elements contained in dual-lens housings 209 and 210. But the magnification levels of optic elements 207 and 208 can be changed in order to adjust the size of the image as it is viewed on a video monitor or display 190. Proximal CF bundle 221 transfers the image from distal end 218 to proximal end 219. Optic elements 207 and 208 have focal lengths that project the image at proximal end 218 to proximal end 213. The image at proximal end 213 couples to color camera 170 using coupler 229.

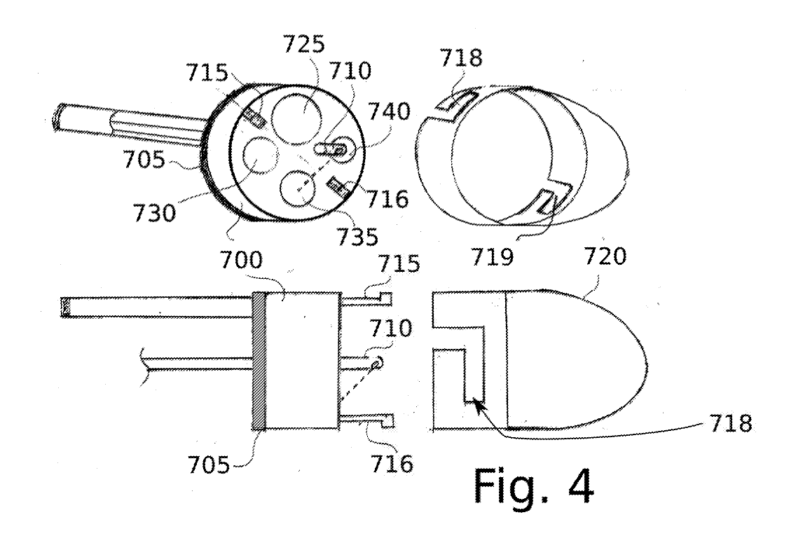

FIG. 4 provides two views of a distal lumen baffle 750 that sits at the distal end endoscopic devices. Cutouts 752, 740, 735 and 730 in baffle 750 are for various lumens that are contained within EN device 110. Baffle 750 is secured at the distal end of EN device 110 by conduit seal 705. The distal end of lens washing system 710 is shown along with two locking pins 715 and 716 that mate with locking slots 718 and 719 to secure the optically transparent shield 720 against baffle 750 when shield 720 is required during a surgical procedure. Of course, one of ordinary skill in the art will recognize that other embodiments exist that use a structure differing from that of distal lumen baffle 750 to provide functionality similar to that of baffle 750.

Also shown in FIG. 2, light system 180 comprises light pipe 182, LED 237, wiring 238, and light pipe tip 248. As with imaging system 160, light system 180 lies within housing 111. Light system 180 generates light, which travels across transmissive joint 155 through conduit 150 and projects past tip 151.

Light pipe tip 248 at the distal end of light pipe 182 has been cut and polished to render light pipe tip 248 non-imaging. In some embodiments this rendition comprises using tip 248 that has been cut and polished to a 30° angle. For purposes of this disclosure, the angle is measured relative to the longitudinal axis of light pipe 182. In other embodiments, this rendition comprises tip 248 that has been cut and polished perpendicular to the longitudinal axis of light pipe 182. An angle of 90° indicates a tip cut perpendicular to the longitudinal axis, and an angle of 30° indicates an angle 30° counterclockwise from the longitudinal axis, in the quadrant between 0° from the axis and perpendicular to the axis.

In some embodiments, light pipe 182 comprises 100 micron stepped-index multimode optical fiber bundles 182A enclosed in a circular close pack configuration at the proximal end, for light coupling efficiency. The fiber bundle passes through the device, then enters the annular gap between two concentric stainless steel hypo tubes. The fibers are arranged in a circular fashion, for uniform light distribution at the distal end of the scope.

In those embodiments that use an LED as the light source, LED 237 generates light that travels through light pipe 182 and projects out of light pipe tip 248 illuminating the region beyond tip 248. Sometimes LED 237 is an OPTEK 1-Watt SMD 6mm rated at 90 luminous Flux (Im). The use of High flux density Luxeon M LED, manufactured by Philips (lumileds). This type of LED has a higher luminous flux, typically 900 (Im), and runs hotter requiring dissipation of the heat. In some embodiments, the electrical input power operates near or above 3 watts.

In some embodiments, EN device 110 has a solid glass waveguide (3.0 mm Dia.), producing an illumination pattern offset from the imaging optical axis. This waveguide is positioned in a side-by-side configuration at the distal end of the scope body. In some embodiments, a fiber bundle is aligned in a circular configuration around the distal imaging lens. This circular configuration surrounding the imaging lens on the scope tip provides a uniform light distribution on the same optical axis as the imaging optics.

Some embodiments use software to connect or remove light reflected into imaging system 160 from body tissue or surgical tools. This software operates in real-time at the receiver end, within 250 milliseconds before being transmitted by the transmitter contained in the devices.

in some embodiments, proximal coherent fiber bundle 221 lacks S-curve, and is straight. Imaging assembly 260 also comprises proximal imaging assembly end 262 that couples to color camera 170 through coupler 229. In the embodiment shown in FIG. 2, the proximal coherent fiber bundle 221 has S-curve 220 near its proximal end.

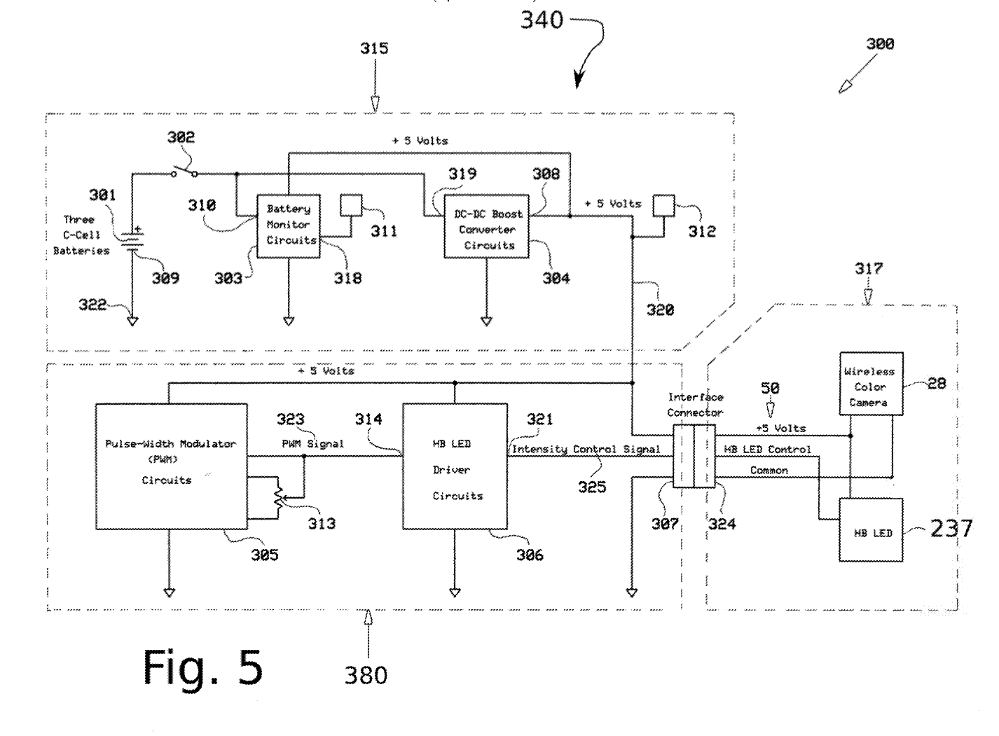

FIG. 5 depicts a block diagram of electrical system 315 that comprises power block 340, camera block 317, and LS controller 380. Power block 340 comprises an energy source such as a battery. Of course, one of ordinary skill in the art will recognize that other embodiments exist that use other types of batteries or that use a power source other than batteries, such as a wall outlet, capacitor-based energy source, or other power source invented in the future. In some self-contained embodiments, the components represented in FIG. 5 are contained within housing 111.

LS controller 380 comprises LED driver circuits 306 and a light source intensity controller 305. Intensity controller 305 and driver circuits 306 receive power from power block 340. Driver circuits 306 modify the power to suit the LED or other light source. And intensity controller 305 adjust the intensity of the light source. Those of ordinary skill in the art are well versed in selecting suitable intensity controllers to match the selected light source.

Referring again to FIG. 5, interface connectors 307 and 324 are two separate components: panel-mount-type connector 307 and mating inline connector 324. Interface connectors 307 and 324 interact with cable 125. Connectors 307 and 324 each have two separate contacts and a common ground; cable 125 is a small diameter, flexible, three-wire cable. Connector 324 is permanently wired to one end of cable 125 while the other end of cable 125 is wired to color camera 170 and LED 237 in handle 140 of EN device 110.

EN device 110 and related invention devices may comprise means for activating a sensor on PCM 120 or related invention devices. The sensor may take the form of a simple switch, or it may take the form of a more complex sensor. For example, the sensor may be a detector that interacts with the means for activating in such a way that the sensor is capable of detecting a unique identifier composing a part of EN device 110 that identifies the origin, manufacturer, and/or type of the endoscopic device. Such an identifier, for example, may send a signal to PCM 120. In another embodiment of the invention, the identifier may include a Radio Frequency Identification (RFID) tag or some other integrated-circuit-based identifier mounted anywhere on or otherwise associated with EN device 110. In another embodiment of the invention, the identifier may include a resistor mounted on the EN device 110. In some of these embodiments, the sensor-identifier interaction causes hardware or software in the PCM 120 to refuse to power EN device 110, such as when the PCM 120 determines that an operator is attempting to inappropriately reuse EN device 120.

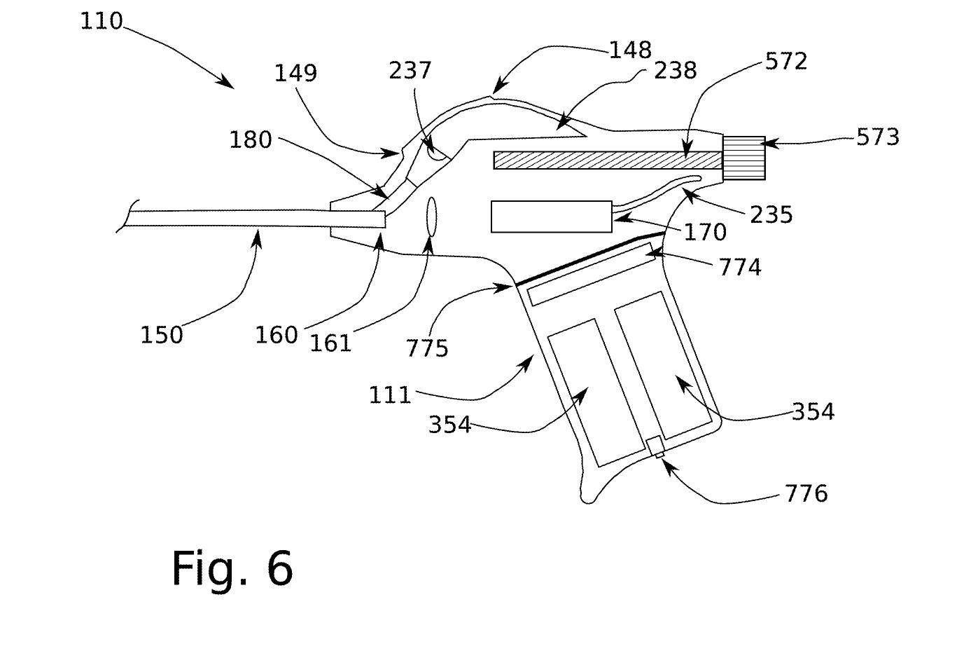

FIG. 6 depicts a self-contained endoscope, otherwise called EN device 110. Housing 111 connects to conduit 150. Imaging system 160 extends through conduit 150 into housing 111. In this case, imaging system 160 comprises proximal achromatic lens 161. Proximal achromatic lens 161 focuses an image transmitted along the conduit imaging system 160 on to color camera 170. Light system 180 also extends through conduit 150 into housing 111. Light system 180 bends out of the path of imaging system 160, once light system 180 enters housing 111. In this embodiment, light system 180 uses coherent optical fibers to transmit light from the housing to the tissue at the distal end of conduit 150. As can be seen, light in this embodiment is produced by LED 237. In some embodiments LED 237 is equipped with finned heat sinks 238 to remove heat that is generated by LED 237. In some embodiments, the anode and cathode connections, such as soldering connections, are optimized to facilitate heat removal, as well. Housing 111 also contains ventilation openings 148 and 149.

Color camera 170 is attached to the focusing mechanism comprising focal assembly adjustment screw 572 and focusing adjustment knob 573. Manipulation of knob 573 causes color camera 170 to move laterally, adjusting the distance between camera 170 and lens 161. This embodiment has optical data processing unit 774 and is powered by batteries 354. The figure shows electrostatic shield 775 disposed between battery 354 and between camera 170 and optical data processing unit 774. Also shown in this figure is antenna 235, which facilitates transmission of optical data from the endoscope to a discrete base unit, and power switch 776.

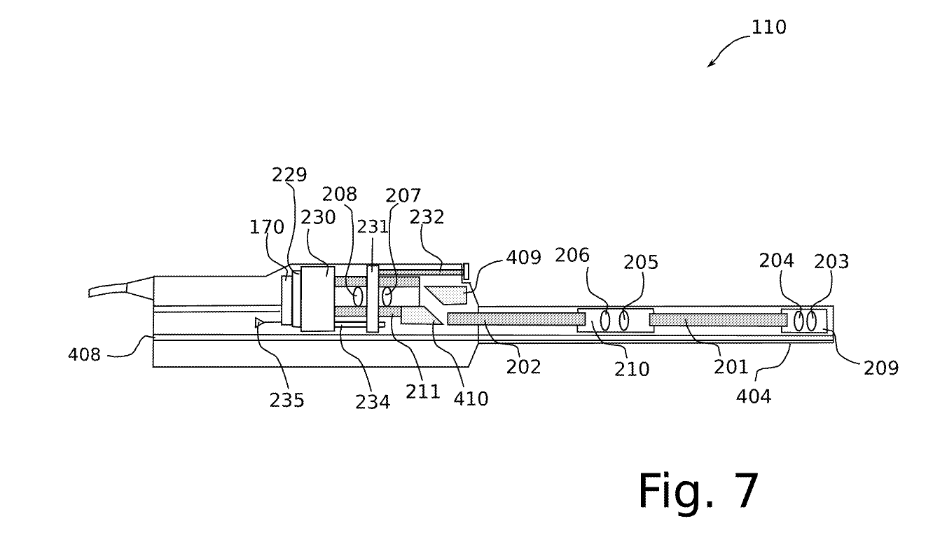

FIG. 7 details a partial assembly of an embodiment of EN device 110 has a dual-folded imaging system 160. The folding occurs within handle 140 and allows EN device 110 to be more compact and allows imaging system 160 to avoid or clear the central axis of EN device 110. The clearance that flows from folding imaging system 160 facilitates a low-friction path through EN device 110, which accepts a surgical device in some embodiments. The surgical device enters the proximal end 411 of EN device 110. In these types of embodiments, color camera 170, coupler 229, focusing mechanism components (230 through 234) and lens housing 211 have been shifted off center of the handle 140. In this embodiment, two 45-degree mirrors 409 and 410, allow folding without substantial degradation of an image.

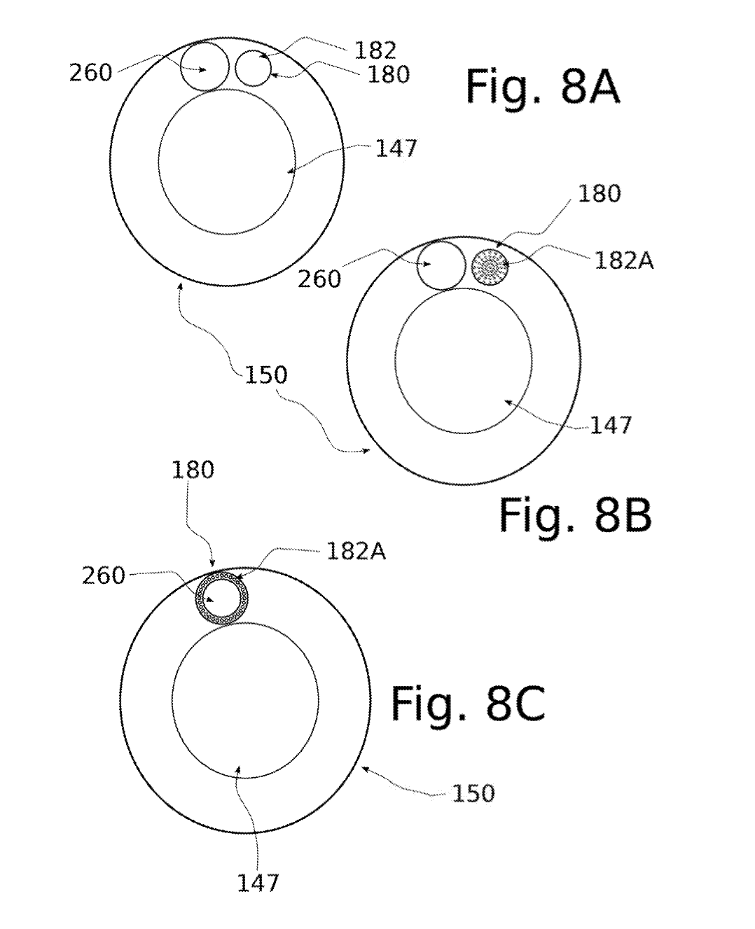

FIGS. 8A-C show various embodiments of conduit 150 in cross-section. FIG. 8A depicts conduit 150 substantially coaxially around tool bore 147. Imaging assembly 260 in this embodiment uses a light pipe for transmitting light representing image data from the distal end of EN device 110. Likewise, light system 180 uses light pipe 182 in this embodiment. Both imaging assembly 260 and light system 180 are sharply offset towards the inner wall of conduit 150 such that both clear the central region leaving space in the central region for tool bore 147.

FIG. 8B depicts conduit 150 substantially coaxially around tool bore 147. Imaging assembly 260 in this embodiment uses a light pipe for transmitting light representing image data from the distal end of EN device 110. Likewise, light system 180 uses coherent fiber bundles made up of optical fibers 182A in this embodiment. Both imaging assembly 260 and light system 180 are sharply offset towards the inner wall of conduit 150 such that both clear the central region leaving space in the central region for tool bore 147.

FIG. 8C shows an embodiment with even more central-region space savings. This figure depicts conduit 150 substantially coaxially around tool bore 147, as before. Imaging assembly 260 in this embodiment uses a light pipe for transmitting light representing image data from the distal end of EN device 110. But in this case, light system 180 is disposed coaxially around imaging assembly 260. As shown, light system 180 uses coherent fiber bundles made up of optical fibers 182A with the individual optical fibers 182A substantially forming a ring around imaging assembly 260. Both imaging assembly 260 and light system 180 continue to be sharply offset towards the inner wall of conduit 150, but in this arrangement use up even less interior space within conduit 150.

Receiver 130 connects to image display 190, which displays the image data. In some embodiments, display 190 displays the image data, displays and records the data, or merely records the data.

In some embodiments, the image clean-up microprocessor will execute a software feature on the receiver end of the hardware package. Without the image clean-up process, the fiber-conduit-based image assembly 260 might exhibits light image artifacts that can be observed under certain conditions.

If an alternative source of relay optical conduits is used such as GRINS, no post imaging processing is needed to remove the artifacts. But generally GRINS are more expensive than coherent fiber bundles.

The small artifacts, caused by the spaces between the drawn optical fibers (^{˜}5-10 microns), can be removed by the use of image processing software, without compromising the integrity of the image.

The image information will be generated within EN device 110 (transmitter) and sent to display 190 or to stand-alone electronic components by wired or wireless transmission methods.

### Operation

In operation, EN device 110 is energized by PCM 120 supplying power through cable 125. Contemporaneously, base 190, wire 200, and receiver 130 are energized. Receiver 130 and color camera 170 establish a wireless data connection with each other. At an appropriate time PCM 120 provides signals to light system 182 to cause appropriate or chosen lighting level to be generated by LED 237. The light from LED 237 travels down light pipe 182 and projects out of light pipe tip 248 illuminating the field adjacent light pipe tip 248. Either before or after turning on light system 180, handle 140, and conduit 150 are inserted into a patient's body, either using or not using a trocar to aid insertion.

EN device 110 projects light from light system 180 onto bodily tissue. That light reflects off of the tissue forming an image.

The image is projected into imaging system 160, as described above. Ultimately, the image impinges on sensor or plate 175, after which, color camera 170 transmits the image data over wireless or wired path to receiver 130. Once the image data is within base 201, the data is displayed on monitor display 190.

For self-contained EN device embodiments similar to those of FIG. 6, in operation the device is set up. Data connectivity between self-contained device 110 through color camera 170 and antennae 235 is established with a base unit having a monitor 190. The device is powered by batteries 354.

Conduit 150 is inserted into the patient, and once conduit is positioned at the desired location, the operator energizes LED 237. Light system 180 projects LED light along light system 180 out of the end of conduit 150, thereby illuminating the internal surgical region. For some high-intensity versions of LED 237, extra heat is conducted away from LED 237 by finned heat sinks 238 and out of housing 111 partially through ventilation openings 148, 149. Light from light system 180 reflects off of the tissue forming an image. The image light enters imaging assembly 260 (part of imaging system 160). The optics of imaging assembly 260 conduct the image light up conduit 150 into housing 111. There, proximal achromatic lens 161 focuses the image light into camera 170 and camera 170 turns the photonic data into electrical data. Within color camera 170 or optical data processing unit 774 various manipulations can be carried out on the image data, as desired. At the desired time, EN device 110 transmits the image data (in some embodiments before or after on-board manipulation) to receiver 130 in the base. There the image can be displayed on monitor or display 190.

When the image data does not arrive at camera 170 in focus, the operator can manipulate knob 573 to bring the image into focus. Rotation of knob 573 causes adjustment screw 572 to rotate. This causes camera 170 to move longitudinally because camera 170 is mounted on screw 572.

In some embodiments, EN device 110 is disassembled after use. For instance, in some embodiments conduit 150 along with imaging system 260 up to lens 161 and along with light system 180 up to just before LED 237 are removed for reconditioning and the remainder of EN Device 110 is discarded. In this type of embodiment, conduit 150 would be cleaned and sterilized and mounted within a new EN device 110. This processing can be carried out at the surgical facility or elsewhere.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true, intended, explained, disclose, and understood scope and spirit of this invention's multitudinous embodiments and alternative descriptions.

Additionally, various embodiments have been described above. For convenience's sake, combinations of aspects composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect. All patents, test procedures, and other documents cited in this specification are fully incorporated by reference to the extent that this material is consistent with this specification and for all jurisdictions in which such incorporation is permitted.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

What is claimed is:

1. An endoscope system comprising a self-contained endoscope having:
a handle;
a conduit having a proximal end connected to the distal end of the handle;
a power and control module disposed within the handle;
a light system disposed within the conduit and within the handle and electrically connected to the power and control module;
an imaging system disposed with in the handle and electrically connected to the power and control module;
and
a video camera disposed within the handle optically connected to the proximal end of the imaging system and electrically connected to the power and control module.

2. The endoscope system of claim 1 wherein the light system comprises coherent fiber bundles.

3. The endoscope system of claim 2 wherein the light system further comprises a high intensity LED.

4. The endoscope system of claim 3 wherein the power and control module comprises a battery.

5. The endoscope system of claim 3 wherein the power and control module comprises a super capacitor.

6. The endoscope system of claim 5 wherein the video camera comprises a wireless receiver or transceiver.

7. The endoscope system of claim 6 wherein the imaging system further comprises an optical data processing unit.

8. The endoscope system of claim 7 wherein the self-contained endoscope further comprises a tool bore disposed at or along the central endoscope axis.

9. The endoscope system of claim 8 wherein the light system is disposed coaxially of the imaging system.

10. The endoscope system of claim 9 wherein the light system path is folded within the handle.

11. The endoscope system of claim 10 wherein the imaging system path is folded within the handle.

12. The endoscope system of claim 11 further comprising a discrete base having a receiver or transceiver and a display.

13. The endoscope of claim 5a wherein the imaging system further comprises an optical data processing unit.

14. The endoscope of claim 13 wherein the imaging system path is folded within the handle.

15. The endoscope of claim 5a wherein the light system path is folded within the handle.

16. The endoscope system of claim 1a further comprising a discrete base having a receiver or transceiver and a display.

17. The endoscope system of claim 16 wherein the light system comprises coherent fiber bundles.

18. The endoscope system of Claim la wherein the light system comprises a high intensity LED and the power and control module comprises a battery.

19. The endoscope system of claim 1a wherein the light system comprises coherent fiber bundles, the power and control module comprises a battery, and wherein the self-contained endoscope further comprises a tool bore disposed at or along the central endoscope axis.

20. An endoscope system comprising a self-contained endoscope having:
a handle;
a conduit having a proximal end connected to the distal end of the handle;
a battery equipped power and control module disposed within the handle;
a light system comprises a coherent fiber bundles and a high intensity LED disposed within the conduit and within the handle and electrically connected to the power and control module and wherein the light system path is folded within the handle;
an imaging system comprising an optical data processing unit disposed within the handle and electrically connected to the power and control module and wherein the imaging system path is folded within the handle;
a video camera having a wireless receiver or transceiver and disposed within the handle optically connected to the proximal end of the imaging system and electrically connected to the power and control module;
a tool bore disposed at or along the central endoscope axis;
and
a discrete base having a receiver or transceiver and a display,
wherein the light system is disposed coaxially of the imaging system.
